(19) United States
(12) Patent Application Publication
(10) Pub. No.: US 2013/0018310 A1
(43) Pub. Date: Jan. 17, 2013

(54) Medicated Module with Time Lock

(71) Applicants: Malcolm Stanley Boyd, Wellesbourne (GB); James Alexander Davies, Leamington Spa (GB); Andrew Gordon Wallace, Highleadon (GB)

(72) Inventors: Malcolm Stanley Boyd, Wellesbourne (GB); James Alexander Davies, Leamington Spa (GB); Andrew Gordon Wallace, Highleadon (GB)

(73) Assignee: SANOFI-AVENTIS DEUTSCHLAND GMBH, Frankfurt am Main (DE)

(21) Appl. No.: 13/520,364

(22) PCT Filed: Feb. 1, 2011
(86) PCT No.: PCT/EP2011/051401

§ 371 Date: Oct. 4, 2012

Related U.S. Application Data
(60) Provisional application No. 61/301,700, filed on Feb. 5, 2010.

(30) Foreign Application Priority Data
Apr. 23, 2010 (EP) 10160842.0

(51) Int. Cl. A61M 5/32; A61M 5/31; A61M 5/50

(52) U.S. Cl. 604/110; 604/191

(57) ABSTRACT

A medicated module (4) for an injection system to co-deliver at least two medicaments is disclosed where a primary delivery device (7) containing a primary medicament accepts a medicated module (4) containing a single dose of a second medicament (2) and where both medicaments are delivered through a single hollow needle (3). The medicated module (4) contains a time lock feature to prevent further use after a predetermined time elapses. The medicated module (4) can also contain a rotary valve (19) operably connected to a retractable needle shield (16) that locks after a predetermined time elapses.

### FIELD OF THE PRESENT DISCLOSURE

Specific embodiments of this disclosure relate to medical devices and methods of delivering at least two drug agents from separate reservoirs using devices having only a single dose setting mechanism and a single dispense interface. A single delivery procedure initiated by the user causes a, preferably, non-user settable dose of a second drug agent and a, preferably, variable set dose of a first drug agent to be delivered to the patient. The drug agents may be available in two or more reservoirs, containers or packages, each containing independent (single drug compound) or pre-mixed (co-formulated multiple drug compounds) drug agents. Specifically, our disclosure concerns a medicated module that has a lock out feature, in particular a time lock, that activates after a predetermined time elapses. The time lock may prevent unintended reuse of the medicated module. This may be of particular benefit where the therapeutic response can be optimized for a specific target patient group, through control and definition of the therapeutic profile.

### BACKGROUND

Certain disease states require treatment using one or more different medicaments. Some drug compounds need to be delivered in a specific relationship with each other in order to deliver the optimum therapeutic dose. Here, combination therapy may be desirable, but not possible in a single formulation for reasons such as, but not limited to, stability, compromised therapeutic performance and toxicology.

For example, in some cases it might be beneficial to treat a diabetic with a long acting insulin and with a glucagon-like peptide-1 (GLP-1), which is derived from the transcription product of the proglucagon gene. GLP-1 is found in the body and is secreted by the intestinal L cell as a gut hormone. GLP-1 possesses several physiological properties that make it (and its analogs) a subject of intensive investigation as a potential treatment of diabetes mellitus.

There are a number of potential problems when delivering two active medicaments or “agents” simultaneously. The two active agents may interact with each other during the long-term, shelf life storage of the formulation. Therefore, it is advantageous to store the active components separately and only combine them at the point of delivery, e.g. injection, needle-less injection, pumps, or inhalation. However, the process for combining the two agents needs to be simple and convenient for the user to perform reliably, repeatedly and safely.

A further problem is that the quantities and/or proportions of each active agent making up the combination therapy may need to be varied for each user or at different stages of their therapy. For example one or more actives may require a titration period to gradually introduce a patient up to a “maintenance” dose. A further example would be if one active requires a non-adjustable fixed dose while the other is varied in response to a patient's symptoms or physical condition. This problem means that pre-mixed formulations of multiple active agents may not be suitable as these pre-mixed formulations would have a fixed ratio of the active components, which could not be varied by the healthcare professional or user.

Additional problems arise where a multi-drug compound therapy is required, because many users cannot cope with having to use more than one drug delivery system or making the necessary accurate calculation of the required dose combination. This is especially true for users with dexterity or cognitive difficulties. In some circumstances it may also be necessary to perform a priming procedure of the device and/or needle cannulae before dispensing the medicaments. Likewise, in some situations, it may be necessary to bypass one drug compound and to dispense only a single medicament from a separate reservoir.

Accordingly, there exists a strong need to provide devices and methods for the delivery of two or more medicaments in a single injection or delivery step that is simple for the user to perform. The above-mentioned problems may be overcome by providing separate storage containers for two or more active drug agents that are then only combined and/or delivered to the patient during a single delivery procedure. Setting a dose of one medicament may automatically fix or determine the dose of the second, preferably non-user settable, medicament. Moreover, the opportunity may be given for varying the quantity of one or both medicaments. For example, one fluid quantity can be varied by changing the properties of the injection device (e.g. dialing a user variable dose or changing the device's “fixed” dose). The second fluid quantity can be changed by manufacturing a variety of secondary drug containing packages with each variant containing a different volume and/or concentration of the second active agent. The user or healthcare professional would then select the most appropriate secondary package or series or combination of series of different packages for a particular treatment regime.

The present disclosure also provides a medicated module that is designed so that a needle guard or shield will lock in a covering or fully extended distal position after a predetermined time has elapsed after the user attaches the medicated module to a drug delivery device and the guard is first retracted. This may prevent unintended reuse of the module while still permitting multiple needle insertions.

These and other advantages will become evident from the following more detailed description of the invention.

### PROBLEM TO BE SOLVED

The problem to be solved by the present invention is to provide a medicated module, a drug delivery device and a needle guard assembly where the administration of a medicament is improved.

### SUMMARY

The disclosed medicated module and drug delivery device may allow complex combinations of multiple drug compounds within a single drug delivery system. In particular, the user may be enabled to set and dispense a multi-drug compound device through one single dose setting mechanism and a single dispense interface. This single dose setter expediently controls the mechanism of the device such that a predefined combination of the individual drug compounds is delivered when a single dose of one of the medicaments is set and dispensed through the single dispense interface. The term drug dispense interface may be, in the context of this disclosure, any type of outlet that allows the two or more medicaments to exit the drug delivery system and be delivered to the patient. In a preferred embodiment the single drug dispense interface comprises a hollow needle cannula.

By defining the therapeutic relationship between the individual drug compounds the drug delivery device may help to ensure that a patient/user receives the optimum therapeutic combination dose from a multi-drug compound device without the inherent risks associated with multiple inputs where the user has to calculate and set the correct dose combination every time he uses the device. The medicaments can be fluids, defined herein as liquids or gases or powders that are capable of flowing and that change shape at a steady rate when acted upon by a force tending to change their shape. Alternatively, one of the medicaments may be a solid that is carried, dissolved or otherwise dispensed with another fluid medicament.

The disclosed medicated module and drug delivery device may be of particular benefit to users with dexterity or cognitive difficulties as the single input and associated predefined therapeutic profile removes the need for them to calculate their prescribed dose every time they use the device and the single input allows considerably easier setting and dispensing of the combined compounds.

In a preferred embodiment, a master drug compound, such as insulin, contained within a multiple dose, user selectable device could be used with a single use, user replaceable, module that contains a single dose of a second medicament and the single dispense interface. When connected to the primary device, the secondary compound is activated/delivered on dispense of the primary compound. Although this disclosure specifically mentions insulin, insulin analogs or insulin derivatives, and GLP-1 or GLP-1 analogs as two possible drug combinations, other drugs or drug combinations, such as an analgesics, hormones, beta agonists or corticosteroids, or a combination of any of the above-mentioned drugs could be used with the present disclosure.

The term “insulin” shall mean insulin, insulin analogs, insulin derivatives or mixtures thereof, including human insulin or a human insulin analogs or derivatives. Examples of insulin analogs are, without limitation, Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin or Des(B30) human insulin. Examples of insulin derivatives are, without limitation, B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-γ-glutamyl)-des(B30) human insulin; B29-N-(N-lithocholyl-γ-glutamyl)-des(B30) human insulin; B29-N-(ω-carboxyheptadecanoyl)-des(B30) human insulin and B29-N-(ω-carboxyheptadecanoyl) human insulin.

As used herein the term “GLP-1” shall mean GLP-1, GLP-1 analogs, or mixtures thereof, including without limitation, exenatide (Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH₂), Exendin-3, Liraglutide, or AVE0010 (H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Ser-Lys-Lys-Lys-Lys-Lys-Lys-NH₂).

Examples of beta agonists are, without limitation, salbutamol, levosalbutamol, terbutaline, pirbuterol, procaterol, metaproterenol, fenoterol, bitolterol mesylate, salmeterol, formoterol, bambuterol, clenbuterol, indacaterol.

Hormones are for example hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

One aspect relates to a time controlled locking mechanism for use with a drug delivery device. The time controlled locking mechanism may be part of or may be integrated in a medicated module. The time controlled locking mechanism may comprise a needle guard. The needle guard may be a sleeve. The needle guard may be adapted and arranged to provide protection against at least one needle cannula. The needle guard may be configured to move in an axial direction during application to an injection site. The time controlled locking mechanism may comprise a time lock assembly. The time lock assembly may be adapted and arranged to prevent movement of the needle guard after a predetermined time elapses. The time lock assembly may be operably connected to the needle guard.

By means of the time controlled locking mechanism, re-use of the needle cannula within the predetermined time may be enabled. This may be especially useful in those cases where the user did not manage to deliver the complete dose in one injection operation, for example. The predetermined time may be chosen such that a misuse of the needle cannula, in particular a use of the needle cannula for different injection operations within a long time period may be prevented.

The time controlled locking mechanism may further comprise a guard lock. The guard lock may be configured to mechanically cooperate with the needle guard. The guard lock may be activatable. When un-activated, the guard lock may not mechanically cooperate with the needle guard. When activated, the guard lock may mechanically cooperate with the needle guard. When activated, the guard lock may prevent the needle guard from moving axially when the needle guard is fully extended distally.

According to an embodiment, the time lock assembly comprises a damper. The damper may be moveable, preferably axially moveable, from a first position to a second position. The damper may be adapted and arranged to activate the guard lock when the damper is in the second position and when the needle guard is fully extended distally.

According to an embodiment, the time controlled locking mechanism comprises a means which is configured to move the damper from the first position to the second position within a predetermined period of time. The means may comprises a gas orifice or a hysteresis member.

According to one aspect, a medicated module is provided. The medicated module may be, preferably releasably, attachable to a drug delivery device. The drug delivery device may comprise a primary reservoir holding one dose, preferably a plurality of doses, of a first medicament. The primary reservoir may be, at least party, filled with the first medicament, before the module is attached to the device. The drug delivery device may be suitable to set and dispense a dose of the first medicament before the medicated module is attached to the device or after the medicated module was removed from the device. Accordingly, the device may be suitable to form a stand-alone device, configured to operate also in absence of the medicated module, for example. For this purpose, a needle cannula may be, preferably removably, attachable to the distal end of the device.

The medicated module may comprise a second medicament, preferably a single dose of the second medicament. The medicated module may comprise a reservoir adapted and arranged for retaining at least one dose, preferably a single dose, of the second medicament. In particular, the reservoir of the medicated module may be filled with second medicament before the medicated module is attached to the drug delivery device, where the medicated module is configured for fluid communication with the primary reservoir. The module reservoir may contain a liquid. In particular, the second medicament may comprise a GLP-1 or a premix of insulin and GLP-1. The reservoir may be annular in shape. The medicated module may be a medicated needle. The medicated module may comprise a first or distal needle cannula. The medicated module may comprise a second or proximal needle cannula. The reservoir of the medicated module may be arranged in the axial direction between the first and the second needle cannula. The module may be adapted and arranged to establish fluid communication between the first needle, the second needle and the reservoir. The distal end of the distal needle cannula may be configured for being applied to an injection site. The proximal end of the distal needle cannula may be configured for piercing the reservoir of the module, in particular a seal or septum arranged at the distal end of the reservoir. The distal end of the proximal needle cannula may be configured for piercing the reservoir of the module, in particular a seal or septum arranged at the proximal end of the secondary reservoir. The proximal end of the proximal needle may be configured for piercing the primary reservoir of first medicament, in particular a seal or septum arranged at the distal end of the primary reservoir. The medicated module may comprise a needle guard. The needle guard may be adapted and arranged to provide protection against at least the first needle cannula. In particular, the needle guard may be adapted for preventing accidental needle sticks. The first needle cannula may be arranged in a portion of the medicated module. The needle guard may be configured to move in an axial direction during application to an injection site. The medicated module may comprise a time lock assembly. The time lock assembly may be operably connected to the needle guard. The time lock assembly may be adapted and arranged to prevent movement of the needle guard after a predetermined time elapses.

The needle guard is preferably configured to move axially, in particular in a distal and a proximal direction, during application to an injection side, in particular when it is pressed against an injection site. When the medicated module is removed or withdrawn from the patient, the needle guard may be returned to its original starting location, e.g. the distal position. The time lock assembly may activate a locking mechanism after a predetermined time elapses, which is measured from the time the medicated module is attached to the drug delivery device and the needle guard is first retracted in a proximal direction. Once locked, the needle guard is prevented from further axial, in particular distal and/or proximal, movement whether attached to the drug delivery device or not. In particular, the needle guard is locked when the needle guard is in the extended, in particular distal, position. Accordingly, the needle guard is preferably prevented from proximal movement by means of the time lock assembly.

Locking of the guard after axial movement can be accomplished in many ways that are known to the art, however, a preferred method includes the use of a moving, rotating or sliding lock contained within the module. This moving lock is configured such that when the time lock is triggered and the predetermined time has expired, the moving lock may be moved to a position that prevents the needle guard from retracting in a proximal direction which is explained later on in more detail.

The medicated module may comprise a housing. The housing may comprise an outer housing. The housing, in particular the outer housing, preferably a proximal end of the outer housing, may be configured for attachment to the drug delivery device. The housing may comprise an inner housing. The reservoir may be arranged in the housing, preferably in the inner housing. The needle guard may be operably connected to the housing, preferably to the inner housing.

The housing may comprise a distal end and a proximal end. The medicated module may comprise the second needle cannula. The previously mentioned first needle cannula may be mounted in the distal end of the housing, in particular of the inner housing. The second needle cannula is expediently mounted in the proximal end of the housing, in particular of the inner housing.

The medicated module may comprise the previously mentioned time lock assembly. The time lock assembly may comprise several mechanical structures and may be configured to activate when the user attaches the medicated module to the drug delivery device and then pushes in the needle guard proximally for the first time. In particular, the time lock assembly may be configured to prevent the needle guard from moving axially after a predetermined time lapses that begins when the needle guard was first retracted, in particular moved proximally, after the medicated module has been attached to the device.

According to an embodiment, the medicated module comprises a guard lock. The activated guard lock may prevent the needle guard from moving axially when the needle guard is fully extended distally.

According to an embodiment, the time lock assembly comprises a damper. The damper may be moveable from a first position to a second position. The damper may be adapted and arranged to activate the guard lock. In particular, the guard lock may be activated when the damper is in the second position and when the needle guard is fully extended distally.

Preferably, a means is provided within the medicated module which is configured to move the damper from the first position to the second position within a predetermined period of time. The means may comprise a gas orifice or a hysteresis member, for example.

According to an embodiment, the time lock assembly comprises a trigger. The time lock assembly may comprise a biasing member. The biasing member may provide a counter force to the force exerted by the means, e.g. the gas orifice or the hysteresis member. Attaching the medicated module first may activate the trigger. The trigger may assert a force on the biasing member, such as a spring or on the previously mentioned hysteresis member. When the needle guard is retracted, this may initiate a mechanical (non-electrical) timing mechanism. This timing mechanism will automatically run for a predetermined time period that may be set by the design and configuration of the time lock assembly, in particular of the gas orifice, the biasing member and/or the hysteresis member.

The size of the orifice that may allow the gas (preferably air) to escape a chamber can be varied to increase or decrease the time period before the lock is activated when the damper moves from the first to the second position. Alternatively or in addition, the design of the biasing member that may be operably connected to the damper can be varied to control the time period. In those designs that may use the hysteresis member, the composition of this member can be selected to vary the restoration time of the member and, thus, the duration of the time lock activation.

According to an embodiment, the medicated module comprises a valve. The valve may comprise a rotary valve. The valve may be operably connected to the needle guard. The valve may be operably connected to the reservoir. The valve, in particular rotation of the valve, may enable fluid communication of the primary reservoir and the reservoir of the medicated module. When the needle guard is pushed into the housing, in particular the outer housing, of the medicated module in the proximal direction, the valve may be activated and may place the second medicament in the medicated module reservoir in fluid communication with two needle cannulae. When the needle guard is fully extended in the distal direction, the valve may be in a closed position and, hence, the two needle cannulae may not be in fluid communication with the reservoir and, hence, with the second medicament.

The medicated module may comprise a bypass. The bypass may comprise a fluid path bypassing the module reservoir. When the needle guard is in the fully extended, i.e. the distal, position, the valve may rotate back placing the first and second needle cannulae in fluid communication with the bypass such that the first medicament from the primary reservoir may flow through both cannula and the bypass without flowing through the module reservoir.

A, preferably single, dose of the second medicament may be contained within the reservoir of the medicated module. Preferably, the reservoir has an annular shape with a central core that comprises part of the inner housing and provides a mount for the needle cannulae. When the needle guard is down, in particular extended in the distal direction, the previously mentioned valve may be aligned with the bypass channel such that none of the second medicament can be dispensed through the attached conduit or second needle cannula. In particular, fluid communication of the primary reservoir and the reservoir of the medicated module may be prevented when the valve is aligned with the bypass channel. The bypass may permit priming of the first/primary medicament contained in the attached drug delivery device at any volume, without dispense of the second medicament. This fluid flow path or channel is used in the priming function of the delivery device. This bypass could be also achieved by a number of other means designed such that the first medicament could flow to the dispense interface without interacting with the second medicament contained within the reservoir of the medicated module and without having to first expel the second medicament.

When the needle guard is retracted, i.e. moved into the module outer housing in a proximal direction, it may be constrained rotationally, but may have a helical feature on the outside that may interface with the module reservoir and may drive, in particular rotate, the valve to an open position. In this open position, the first and a second cannula may now be in fluid communication with the single dose of the second medicament and the flow path may run through the module reservoir. The medicated module may stay in this position until the needle guard is moved distally, at which point the axial movement of the needle guard may switch the valve back to the bypass channel.

A further aspect relates to a drug delivery device or system. The drug delivery device may be configured to deliver two or more medicaments. The drug delivery device may comprise a housing. The housing may comprise a primary reservoir of a first medicament containing at least one drug agent, preferably a plurality of doses of the drug agent. The drug delivery device may comprise the previously described medicated module. The medicated module may be configured for fluid communication with the primary reservoir of the device.

According to an embodiment, connection of the medicated module to the housing of the device and retraction of the needle guard, in particular retraction of the needle guard in the proximal direction for the first time after attachment of the medicated module to the device, triggers the time lock. In particular the previously mentioned damper may be enabled to move from the first into the second position to activate the previously described guard lock for preventing movement of the needle guard after a predetermined period of time when the needle guard is in the fully extended position. Furthermore, retraction of the needle guard in the proximal direction may operate, in particular open, the valve such that fluid communication of the primary reservoir and the reservoir of the medicated module may be enabled.

According to an embodiment, the device is operable through a single dose setter and a single dispense interface. The single dose setter is preferably comprised in the housing of the device. The single dose setter may be operably connected to the primary reservoir of the first medicament. The device comprises the single dispense interface configured for fluid communication with the primary reservoir and the attached medicated module. The device may comprise a dose button. The dose button may be operably connected to the primary reservoir of the first medicament. The dose button may be adapted and arranged to activate a dose delivery action when pushed by a user.

The present disclosure also covers a method of dispensing a fixed dose of one, preferably the second, medicament and a variable dose of one, preferably the first/primary, medicament from separate reservoirs that involves the steps of first attaching a medicated module to a delivery device wherein the attachment activates the previously mentioned trigger that may initiate a first step of a mechanical timer that will allow the needle guard to perform multiple retractions within a predetermined time interval if required. A predetermined time period may start when the needle guard is retracted for the first time and the medicated module is attached to the drug delivery device. During this predetermined time period the user can prime the device using the first medicament while bypassing the single dose of the second medicament contained in the module reservoir. If the user has not already set a dose of the primary/first medicament, the user may then set a dose of the primary/first medicament contained in the primary reservoir of the drug delivery device using the single dose setter. Application of the medicated module to the patient's injection site causes the needle guard to retract proximally activating the valve, preferably the rotary valve, that may place the second medicament in fluid communication with the two needle cannulae and the reservoir of first medicament contained in the primary reservoir of the delivery device. In this position, the set dose of first medicament will flow through the module reservoir and flush/push out the single dose of the second medicament.

With a single activation of the dose button, when the needle guard is retracted, the medicament from the primary reservoir and the second medicament from the medicated module can be expelled through the second needle cannula. Upon completion of the delivery procedure, substantially all of the second medicament may have been expelled as well as the selected dose of the first medicament through the single dispense interface. By “substantially all” we mean that at least about 80% of the second medicament is expelled from the drug delivery device, preferably at least about 90% is expelled. Additionally, if more of the first medicament needs to be injected, another dose can be set and injected before the time lock activation time expires, thus preventing the guard from retracting too early.

According to a preferred embodiment, a time controlled locking mechanism for use with a drug delivery device is provided. The time controlled locking mechanism comprises a needle guard. The needle guard is adapted and arranged to provide protection against at least one needle cannula. The needle guard is configured to move in an axial direction during application to an injection site. The time controlled locking mechanism comprises a time lock assembly which is adapted and arranged to prevent movement of the needle guard after a predetermined time elapses. The time lock assembly is operably connected to the needle guard.

According to a preferred embodiment a medicated module is provided, the medicated module being attachable to a drug delivery device, the drug delivery device comprising a primary reservoir of a first medicament and the medicated module comprising a second medicament. The medicated module comprises a reservoir adapted and arranged for retaining at least one dose of the second medicament and a needle guard adapted and arranged to provide protection against at least a first needle cannula arranged in a portion of the medicated module. The needle guard is configured to move in an axial direction during application to an injection site. The medicated module comprises a time lock assembly adapted and arranged to prevent movement of the needle guard after a predetermined time elapses, wherein the time lock assembly is operably connected to the needle guard.

According to a preferred embodiment, a medicated module is provided, the module being attachable to a drug delivery device, and the module comprising an outer housing having a connector configured for attachment to a drug delivery device and an inner housing having a proximal end and a distal end. The module further comprises a reservoir in the inner housing comprising a single dose of a medicament, a guard operably connected to the inner housing and configured to move in an axial direction during application to an injection site and a valve operably connected to the guard and the reservoir.

According to a preferred embodiment, a medicated module is provided, the module being attachable to a drug delivery device, and the module comprising, an outer housing having a connector configured for attachment to a drug delivery device and an inner housing having a proximal end and a distal end. The module further comprises a reservoir in the inner housing comprising a single dose of a medicament, a guard operably connected to the inner housing and configured to move in an axial direction during application to an injection site a time lock assembly operably connected to the guard.

The medicated module can be designed for use with any drug delivery device with an appropriate compatible interface. However, it may be preferable to design the module in such a way as to limit its use to one exclusive primary drug delivery device (or family of devices) through employment of dedicated or coded features to prevent attachment of a non-appropriate medicated module to a non-matching device. In some situations it may be beneficial to ensure that the medicated module is exclusive to one drug delivery device while also permitting the attachment of a standard drug dispense interface to the device, which could include a standard type A needle interface or a standard ‘zero dose’ safety guard needle with a compatible interface. This would allow the user to deliver a combined therapy when the module is attached, but would also allow delivery of the primary compound independently through a standard drug dispense interface in situations, such as, but not limited to, dose splitting or top-up of the primary compound.

The medicated module makes it expediently possible to tailor dose regimes when required, especially where a titration period is necessary for a particular drug. The medicated module could be supplied in a number of titration levels with obvious differentiation features such as, but not limited to, aesthetic design of features or graphics, numbering etc, so that a patient could be instructed to use the supplied medicated module in a specific order to facilitate titration. Alternatively, the prescribing physician may provide the patient with a number of “level one” titration medicated modules and then when these were finished, the physician could then prescribe the next level. A key advantage of this titration program is that the primary device remains constant throughout.

According to a preferred embodiment, a drug delivery system is provided to deliver two or more medicaments operable through a single dose setter and a single dispense interface. The drug delivery system comprises a housing containing a single dose setter operably connected to a primary reservoir of a first medicament containing at least one drug agent. The drug delivery system comprises a dose button operably connected to the primary reservoir of medicament. The drug delivery system comprises a medicated module configured for fluid communication with the primary reservoir, where the medicated module comprises a module outer housing having a connector configured for attachment to the housing and a module inner housing having a proximal end and a distal end housing, a secondary reservoir in the module inner housing comprising a single dose of a second medicament, a guard operably connected to the module inner housing and configured to move in an axial direction during application to an injection site, a rotary valve operably connected to the guard and the secondary reservoir and a time lock assembly operably connected to the guard. Connection of the medicated module to the housing and retraction of the guard in a proximal direction triggers the time lock and operates the rotary valve.

According to an embodiment, the primary reservoir contains a liquid medicament. The drug agent in the primary reservoir may comprise insulin. According to an embodiment, the secondary reservoir contains a liquid medicament. The secondary medicament may comprise a GLP-1. The secondary medicament may comprise a premix of insulin and a GLP-1.

According to a preferred embodiment a needle guard assembly is provided for attachment to a drug delivery device, the needle guard assembly comprising an outer housing having a connector configured for attachment to a drug delivery device and an inner housing having a proximal end and a distal end. The needle guard assembly comprises at least one needle cannula connected to either the inner or outer housings, a guard operably connected to the inner housing and configured to move in an axial direction during application to an injection site and a time lock operably connected to the guard. Said needle guard assembly may comprise all features previously described in connection with the medicated module.

In a preferred embodiment, the primary drug delivery device is used more than once and therefore is multi-use. However, the drug delivery device may also be a single use disposable device. Such a device may or may not have a replaceable reservoir of the primary drug compound, but the present disclosure is equally applicable to both scenarios. It is also possible to have a suite of different medicated modules for various conditions that could be prescribed as one-off extra medication to patients already using a standard drug delivery device. Should the patient attempt to reuse a previously used medicated module, the locking needle guard may be activated after the predetermined time period of the mechanical time lock assembly has expired. Other means of alerting the user may include some (or all) of the following:
Physical prevention of medicated module re-attachment to the primary drug deliver device once the module has been used and removed.
Physical/hydraulic prevention of subsequent liquid flow through the drug dispense interface once it has been used.
Physical locking of the dose setter and/or dose button of the primary drug delivery device.
Visual warnings (e.g. change in color and/or smell and/or warning text/indicia within an indication window on the module once insertion and/or fluid flow has occurred).
Tactile feedback (presence or absence of tactile features on the outer surface of the module hub following use).

A further feature of this embodiment is that both medicaments may be delivered via one injection needle and in one injection step. This offers a convenient benefit to the user in terms of reduced user steps compared to administering two separate injections. This convenience benefit may also result in improved compliance with the prescribed therapy, particularly for users who find injections unpleasant or who have cognitive or dexterity difficulties.

These as well as other advantages of various aspects of the present invention will become apparent to those of ordinary skill in the art by reading the following detailed description, with appropriate reference to the accompanying drawings.

The scope of the invention is defined by the content of the claims. The invention is not limited to specific embodiments but comprises any combination of elements of different embodiments. Moreover, the invention comprises any combination of claims and any combination of features disclosed by the claims.

### BRIEF DESCRIPTION OF THE DRAWINGS

Exemplary embodiments are described herein with reference to the drawings, in which:

FIG. 1 illustrates a perspective view of one possible drug delivery device.

FIG. 2 illustrates a cross-sectional view of an embodiment of the medicated module having a mechanical time lock assembly.

FIG. 3 illustrates the embodiment of the medicated module of FIG. 2 where the medicated module is attached to a drug delivery device.

FIG. 4 illustrates a perspective view of one component of a preferred time lock mechanism.

FIG. 5 shows a cross-section view of the time lock features and the rotary valve.

FIG. 6 shows the internal mechanism a preferred embodiment of the time lock.

FIG. 7 shows the internal mechanism of a preferred embodiment of the time lock.

FIG. 8 shows the internal mechanism cross-section view of a preferred embodiment of the time lock when the needle guard is retracted.

FIG. 9 shows the internal mechanism a preferred embodiment of the time lock where the guard is covering the needle.

FIG. 10 shows a cross-section view of another preferred embodiment of the time lock.

### DETAILED DESCRIPTION

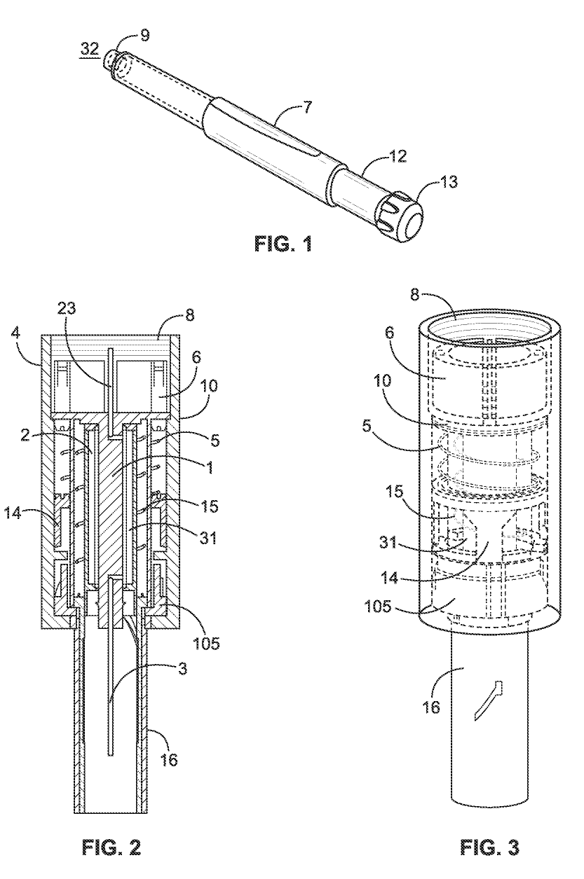

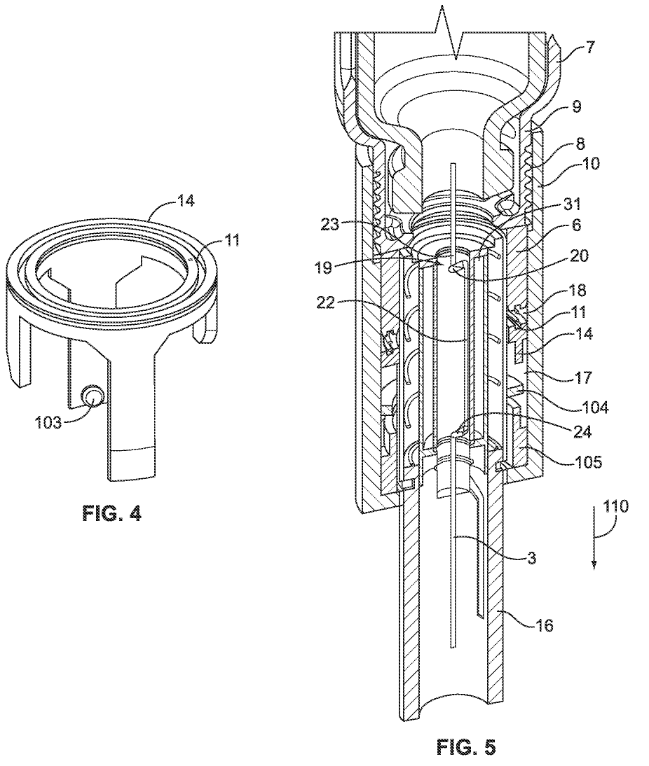

Specific embodiments of the disclosed medicated module and drug delivery device enable administering a fixed predetermined dose of a second drug compound (medicament) and a variable dose of a primary or first drug compound through a single output or drug dispense interface. Setting the dose of the primary medicament by the user is independent of a single dose of the second medicament, which preferably is contained in a reservoir in a medicated module that is attachable to a drug delivery device. In a preferred embodiment, the drug dispense interface is a needle cannula (hollow needle). FIG. 1 illustrates one example of a drug delivery device 7. A medicated module 4 (see FIGS. 2-5) can be attached to a connection means 9 arranged at the distal end 32 of the device 7. Each medicated module 4 is preferably self-contained and provided as a sealed and sterile disposable module that has an attachment means 8. Attachment means 8 is compatible to the attachment means 9 at the distal end 32 of device 7. Although not shown, the medicated module 4 could be supplied by a manufacturer contained in a protective and sterile container, where the user would peel or rip open a seal or the container itself to gain access to the sterile medicated module 4. In some instances, it might be desirable to provide two or more seals for each end of the medicated module 4.

Any known attachment means 8 can be used to attach the medicated module 4 to the chosen drug delivery device 7, including all types of permanent and removable connection means, such as threads, snap locks, snap fits, luer locks, bayonet, snap rings, keyed slots, and combinations of such connections. FIG. 2 illustrates the attachment means 8 as a thread that would engage like threads 9 of the distal end 32 of drug delivery device 7. The embodiments shown in the figures have the benefit of the second medicament 2 as a single dose being contained entirely within an annular reservoir 31, hence minimizing the risk of material incompatibility between the second medicament 2 and the materials used in the construction of the medicated module 4, specifically central core or inner housing 1 (see FIG. 2) or any of the other parts used in the construction of the medicated module 4.

To minimize the residual volume of the second medicament 2, caused by recirculation and/or stagnant zones, that might remain in reservoir 31 at the end of the dispense operation, it is preferable to have the reservoir 31 configured or designed to maximize the amount of medicament dispensed. A preferred shape is the annulus as shown in the figures. Additionally or alternatively, a flow distributor system could be configured to fit within the annular space or the reservoir 31 itself could be configured to include flow distribution features that would ensure the maximum amount of the second medicament 2 is expelled from the reservoir 31. Preferably, the design of such a flow distribution system should ensure that at least about 80% of the second medicament 2 is expelled from the module reservoir 31 through the distal end of a needle 3. Most preferably, at least about 90% should be expelled. Ideally, displacement of the first medicament from the primary reservoir in the drug delivery device 7 through the module reservoir 31 will displace the second medicament 2 without substantial mixing of the two medicaments.

Attachment of the medicated module 4 to the multi-use device 7 sets or energizes a time lock mechanism contained within the medicated module 4 by distal movement of a trigger 6. Referring to the embodiment shown in FIGS. 2-5, the time lock comprises a biasing member, preferably a spring 5, a damper 14, and a gas orifice 11. Prior to attachment to a cartridge holder of the device 7, the springs 5 and 15 within the module 4 are in a relaxed state. Spring 15 is operably connected to needle guard 16 and forces the guard 16 downwardly in a distal direction to safely cover the tip of needle 3. Spring 5 is operably connected to the damper 14 on its distal end and to trigger 6 on its proximal end. Trigger 6 interfaces with connector 9 of the drug delivery device 7 when the medicated module 4 is attached. In the embodiment illustrated, trigger 6 is forced in the distal direction 110 when the module 4 is attached to the delivery device 7, hence compressing spring 5 and thus exerting a force on damper 14. A ratchet, clip, snap lock or other locking feature(s) (not shown) on the trigger 6 prevent the trigger 6 from returning to the starting position and, thus, spring 5 is maintained in a compressed condition until the needle guard 16 is retracted. As such, spring 5 can only relax in one direction, e.g. the distal direction, after the first connection to the drug delivery device 7.

FIG. 5 shows spring 5 in the compressed state after attachment of the module 4 to the injection device 7. Before the needle guard 16 is pressed or applied to an injection site to cause it to move in the proximal or retraction direction, the reservoir 31 is in a priming position where the primary medicament contained in device 7 can be used to prime the module 4. In this first or priming position the reservoir 31 is rotational oriented such that fluid (primary medicament) can flow through needle cannula 23 in a bypass route without making fluid contact with the second medicament 2 in the reservoir 31 exiting through needle cannula 3. The second medicament 2 remains sealed within reservoir 31.

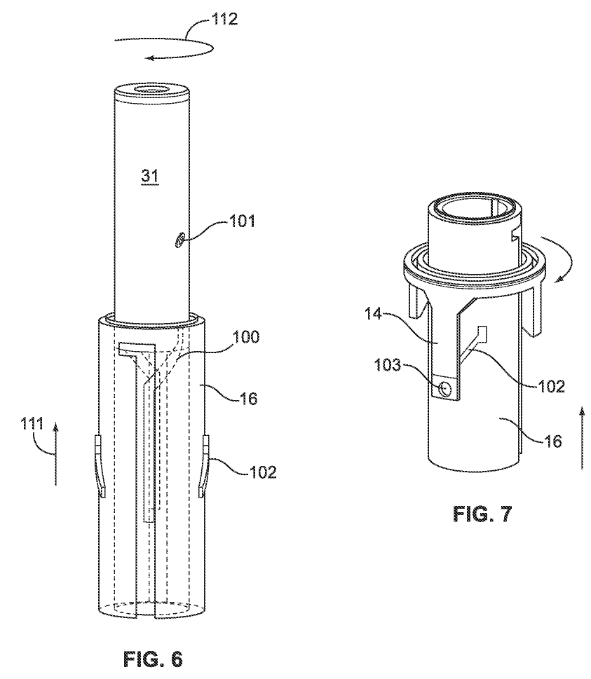

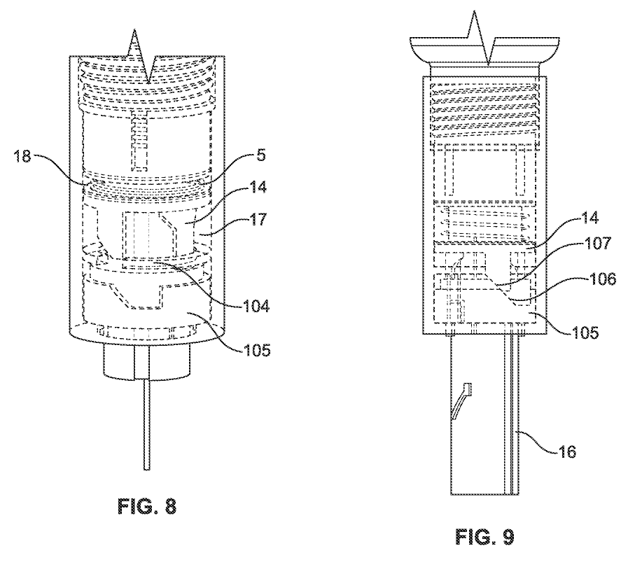

When the needle guard 16 is moved in the proximal or retraction direction (opposite to distal direction 110), it causes the reservoir 31 to rotate such that medicament flow route is altered from the priming position to a dose delivery position. FIG. 6 illustrates the retraction 111 of the needle guard 16 and the rotational movement 112 of the reservoir 31 caused by the interaction of the slot profile 100 and protrusion 101 on reservoir 31. As the needle guard 16 retracts (see FIG. 7), ramp 102 interacts with tab 103 on damper 14 causing it to rotate relative to a stop feature 104 on the inside of the outer housing 10. FIG. 8 illustrates this rotation and movement relative to the stop feature 104. With the engagement of the stop feature 104 and damper 14 being removed, the damper 14 can move in the distal direction due to the force exerted by spring 5. This starts the timer feature of the time lock mechanism. Because the damper 14 is slidably sealed to the inner surface of the outer housing 10, the only way air can pass from compartment 17 to compartment 18 (see FIG. 8) is through a very small aperture or orifice 11. This restriction of the air flow provides a counter force to the force exerted by spring 5 and results in the damper 14 gradually moving downwards (distally) under the force of spring 5. The size of the orifice 11 can dictate the speed of movement of the damper 14 and, thus, the length of time before the module 4 is locked from further use. Until the damper 14 is in its final position, before needle guard 16 retraction is blocked, the user can retract the needle guard 16 multiple times to make multiple injections. When the damper 14 is in its final position and the needle guard 16 is in its extended, e.g. distal, position the guard 16 will be locked and prevented from moving in the proximal direction. This can be accomplished in a number of ways that are not critical to the present disclosure. For example, as illustrated in FIGS. 2-9 one embodiment uses a rotating lock 105. As shown in FIG. 9, as the damper 14 begins to move distally it operably interacts through ramp features 106 and 107 on rotating lock 105 causing it turn in a manner that locks the needle guard 16 from retraction when in the fully extended position. At the end of the timed period, as soon as the needle guard 16 reaches the bottom of its travel the rotating needle guard lock can complete its rotation. During the final rotation, a tab or other protrusion or snap lock on the rotating lock 105 can move into position, such as a circumferential groove in the needle guard 16, which prevents any further needle guard 16 axial movement. The needle guard 16 is now locked out. Preferably, the tab can only rotate and lock out the needle guard 16 when the needle guard 16 is fully down. The design is such that if the needle guard 16 is retracted when the timer runs out, the device 7 will only lock out once the needle guard 16 has moved back down to the safe position. The force of the spring 15 causes the rotating needle guard lock to move into the lock position once the needle guard 16 is fully extended. The spring 5 does not fully relax because there must be enough force to cause the final rotation of the rotating needle guard lock resulting in the device 7 locking out.

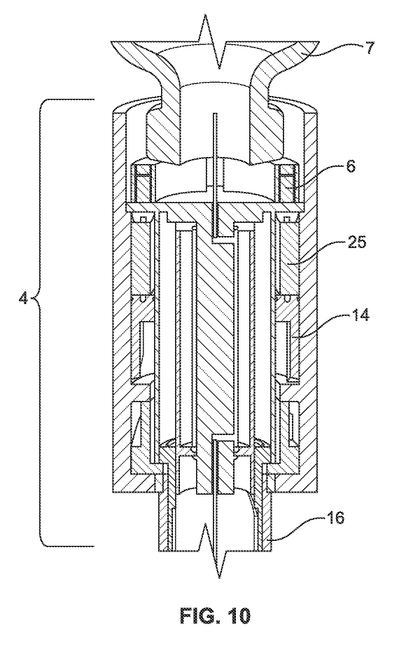

FIG. 10 shows an alternative embodiment of the time lock mechanism of the present disclosure where spring 5 and orifice 11 are replaced with hysteresis material 25. The hysteresis material 25 can be any material that can be compressed and eventually will expand back to its original shape over a specific time period. Trigger 6 compresses hysteresis material 25 when the medicated module 4 is attached to delivery device 7. Once needle guard 16 is retracted, the hysteresis material 25 can then start to expand and exert a force on damper 14, which moves in the distal direction to activate a guard lock. The unlocking of the hysteresis material 25 can be accomplished as described above where the retraction of the needle guard 16 cause the damper 14 to rotate and disengage from a stop member. When the hysteresis material 25 has fully expanded and the guard 16 has achieved its fully extended position, it will be locked from further retraction.

The medicated module 4 comprises two needle cannulae 3, 23. The first needle cannula 3 is arranged at the distal end of the module 4. The second needle cannula 23 is arranged proximally from the first needle cannula 3. The reservoir 31 is axially arranged between the needle cannulae 3, 23. The first and the second needle cannulae 3, 23 are positioned to establish fluid communication with the reservoir 31.

The medicated module 4 also has a valve 19 that is activated when the needle guard 16 is retracted. The valve 19 places the medicament 2 in the reservoir 31 in fluid communication with needle cannulae 3 and 23 when the guard 16 is in the retracted or proximal position. Prior to the retraction of the guard 16, the valve 19 is in a first position where the needle cannulae 3, 23 are in fluid communication with a bypass 22. FIG. 5 shows one possible embodiment of this valve 19, that being a rotary valve 19, positioned in the bypass position. Needle cannula 23 is in fluid communication with channel 20 at its distal end and is in fluid communication with the primary medicament in the delivery device 7 when the medicated module 4 is attached. Outlet needle cannula 3 is in fluid communication with channel 24. When valve 19 is in the bypass position, the channels 20 and 24 are in fluid communication with bypass channel 22. This position allows primary medicament to flow through needle 23, through channel 20, down bypass channel 22, out through channel 24, and finally exiting needle 3. This valve position completely isolates the second medicament 2 in reservoir 31, thus preventing it from exiting needle 3.

When valve 19 is turned to its second position, as described below, channels 20 and 24 become connected directly with reservoir 31 and the second medicament 2 contained therein. In this valve position the medicament from the drug delivery device 7 is now in fluid communication with reservoir 31 and upon activation of a dose button 13, the primary medicament will force the second medicament 2 out of the reservoir 31 into outlet needle 3. Rotation of valve 19 is caused when the needle guard 16 is moved in the proximal direction. As the guard 16 retracts, the reservoir 31 is rotated from the bypass position to an inject position. Channels 20 and 24 remain stationary. Although there are many ways to cause the reservoir 31 to rotate, one method includes the use of a helical path or groove on the guard 16 that interacts with a protrusion or rib on the reservoir 31. Constraining rotation of the guard 16 as it retracts will cause the reservoir 31 to track in the helical groove and rotate as the rib follows the path. When the guard 16 moves in the opposite direction (distally) as it extends out of the module housing 10, the reservoir 31 and the valve 19 will move back to the bypass position.

Once the medicated module 4 is attached to the drug delivery device 7, the user can prime the system using any amount of the primary medicament and then perform an injection via activation of dose button 13 on device 7. The dose button 13 can be any triggering mechanism that causes the dose of the first medicament that was set by a dose setter 12 to move towards the distal end 32 of the device 7. In a preferred embodiment the dose button 13 is operably connected to a spindle that engages a piston in the primary reservoir of the first medicament.

The guard or safety shield 16 could be any design that would prevent accidental needle sticks and/or reduce the anxiety experienced by users who suffer from needle phobia. The exact design of the safety shield 16 is not critical to the present disclosure, however, a preferred design, as disclosed above, is one which locks out after a predetermined amount of time having been activated by attachment to a drug delivery device 7 and then subsequently triggered on retraction of the needle guard 16. A user can retract the needle guard 16 any number of times within the time-lock period, but after that period the device is locked. The device 7 works for single insertion or multiple insertions as long as those other than the first insertion are carried out within the allowed time period. In a preferred embodiment, the locking of the needle guard 16 will cause or trigger a display feedback to the user, for example, change in color, tactile, audible, or the like, that indicates the guard is lock and can no longer be retracted. Additionally, user feedback can be included to provide the user with an estimation of how much time is remaining before the needle guard is locked out. This could be accomplished with an electronic timer connected to sensors, color change, audible clicking, or the like operably connected to the movement of the damper, rotating lock or both.

In any of the above described embodiments the second medicament 2 in the medicated module 4 may be either in a powdered solid state, any fluid state contained within the reservoir 31, or coated to the inside surface of the drug dispense interface 3. The greater concentration of the solid form of the medicament 2 has the benefit of occupying a smaller volume than the liquid having lower concentration. This in turn reduces the ullage of the medicated module 4. An additional benefit is that the solid form of the second medicament 2 is potentially more straightforward to seal in the reservoir 31 than a liquid form of the medicament 2. The device 7 would be used in the same manner as the preferred embodiment with the second medicament 2 being dissolved by the first medicament during dispense.

As mentioned, to minimize diffusion of the second medicament 2 contained in the reservoir 31 within the medicated module 4 into the primary medicament during dispense of the medicaments, a flow distributor system can be incorporated into the reservoir 31. This flow distributor also ensures efficient expulsion of the second medicament 2 from the system and greatly minimizes residual volume. One possible embodiment of the flow distributor is an annular insert that could be positioned in the annular reservoir 31 and configured such that the second medicament 2 fills flow channels that are defined by the shape and location of one or more support ribs. The flow distributor can be constructed of any material that is compatible with the primary and second medicaments. A preferred material would be that typically used to manufacture septa or pistons (bungs) found in multi-dose medicament cartridges, although any material that is compatible with the medicament during long term storage would be equally applicable. The shape of the flow channels can be optimized for a plug flow of medicament by varying the dimensions and number of support ribs. The cross-sectional area of the annulus formed between the flow distributor and the wall of the reservoir 31 should be kept relatively small. The volume available to store the second medicament 2 would equal the internal volume of the reservoir 31 minus the volume of the flow distributor. Therefore, if the volume of the flow distributor is marginally smaller than the internal volume of the reservoir 31, a small volume is left which the second medicament 2 occupies. Hence, the scale of both the reservoir 31 and the flow distributor can be large while storing a small volume of medicament 2. A further benefit of this is that as the available volume for medicament 2 is defined by the difference in volumes between the flow distributor and its housing, the external reservoir geometry is not dictated by the volume of medicament 2. Accordingly, for small volumes of second medicament 2 (e.g. 25-50 micro liters) the reservoir 31 can be of an acceptable size for handling, transport, manufacture, filling and assembly.

The connection or attachment between the medicated module 4 of the above described embodiments may contain additional features (not shown), such as connectors, stops, splines, ribs, grooves, and the like design features, that ensure that specific medicated modules 4 are attachable only to matching drug delivery devices 7. Such additional features would prevent the insertion of a non-appropriate medicated module 4 to a non-matching injection device 7.

The shape of the medicated module 4 may be a cylindrical body or any other geometric shape suitable for defining a fluid reservoir 31 or for containing discrete self-contained reservoir 31 of the second medicament 2 and for attaching one or more needle cannulae 3, 23. The reservoir 31 can be manufactured from glass or other drug contact suitable material. The integrated injection needle 3 can be any needle cannula suitable for subcutaneous or intramuscular injection. Preferably, the medicated module 4 is provided by a drug manufacturer as a stand-alone and separate device that is sealed to preserve sterility. The sterile seal of the module 4 is preferably designed to be opened automatically, e.g. by cutting, tearing or peeling, when the medicated module 4 is advanced or attached to the drug delivery device 7 by the user but any other means of sterile supply are also equally applicable and acceptable.

The medicated module 4 should be designed to operate in conjunction with a multiple use injection device 7, preferably a pen-type multi-dose injection device, similar to what is illustrated in FIG. 1. The injection device 7 could be a reusable or disposable device. By disposable device it is meant an injection device that is obtained from the manufacturer preloaded with medicament and cannot be reloaded with new medicament after the initial medicament is exhausted. The device 7 may be a fixed dose or a settable dose and preferably a multi-dose device, however, in some cases it may be beneficial to use a single dose, disposable device.

A typical injection device contains a cartridge or other reservoir of medication. This cartridge is typically cylindrical in shape and is usually manufactured in glass. The cartridge is sealed at one end with a rubber bung and at the other end by a rubber septum. The injection device is designed to deliver multiple injections. The injection device may further comprise a dose setter; the dose setter may be operably connected to the reservoir. The injection device comprises a dose button; the dose button may be operably connected to the reservoir. The dose button may be any triggering mechanism that causes the dose of the medicament that was set by the dose setter to move distally towards the distal end of the device. In a preferred embodiment, the dose button is operably connected to a spindle that engages a piston in the reservoir. In a further embodiment the spindle is a rotatable piston rod comprising two distinct threads. The delivery mechanism is typically powered by a manual action of the user, however, the injection mechanism may also be powered by other means such as a spring, compressed gas or electrical energy.

Exemplary embodiments of the present invention have been described. Those skilled in the art will understand, however, that changes and modifications may be made to these embodiments without departing from the true scope and spirit of the present invention, which is defined by the claims.

### REFERENCE NUMERALS

1 Inner housing
2 Second medicament
3 Needle
4 Medicated module
5 Spring
6 Trigger
7 Drug delivery device
8 Attachment means
9 Connection means
10 Outer housing
11 Gas orifice
12 Dose setter
13 Dose button
14 Damper
15 Spring
16 Needle guard
17 Compartment
18 Compartment
19 Valve
20 Channel
22 Bypass channel
23 Needle cannula
24 Channel
25 Hysteresis material
31 Reservoir
32 Distal end
100 Slot profile
101 Protrusion
102 Ramp
103 Tab
104 Stop feature
105 Guard lock/rotating lock
106 Ramp feature
107 Ramp feature
110 Distal direction
111 Retraction
112 Rotational movement

1. A time controlled locking mechanism for use with a drug delivery device, comprising
a needle guard adapted and arranged to provide protection against at least one needle cannula, wherein the needle guard is configured to move in an axial direction during application to an injection site; and
a time lock assembly adapted and arranged to prevent movement of the needle guard after a predetermined time elapses, wherein the time lock assembly is operably connected to the needle guard.

2. The time controlled locking mechanism according to claim 1, further comprising a guard lock where the activated guard lock prevents the needle guard from moving axially when the needle guard is fully extended distally.

3. The time controlled locking mechanism according to claim 2, where the time lock assembly comprises a damper being moveable from a first position to a second position, wherein the damper is adapted and arranged to activate the guard lock when the damper is in the second position and when the needle guard is fully extended distally.

4. The time controlled locking mechanism of claim 3, comprising a means which is configured to move the damper from the first position to the second position within a predetermined period of time.

5. The time controlled locking mechanism of claim 4, wherein the means comprises a gas orifice or a hysteresis member.

6. A medicated module attachable to a drug delivery device, the drug delivery device comprising a primary reservoir of a first medicament and the medicated module comprising a second medicament, wherein the medicated module comprises a reservoir adapted and arranged for retaining at least one dose of the second medicament, and wherein the medicated module comprises the time controlled locking mechanism according to claim 1.

7. The medicated module according to claim 6, where the needle guard is prevented from moving axially after a predetermined time lapses that begins when the needle guard is first retracted after the medicated module has been attached to the drug delivery device.

8. The medicated module according to claim 6, comprising a valve which is operably connected to the needle guard and the reservoir and which is configured to enable fluid communication of the primary reservoir with the reservoir of the medicated module.

9. The medicated module according to claim 6, comprising a housing configured for attachment to the drug delivery device wherein the reservoir is arranged in the housing, and wherein the needle guard is operably connected to the housing.

10. The medicated module according to claim 9, wherein the housing comprises a distal end and a proximal end, and wherein the medicated module comprises a second needle cannula, where the second needle cannula is mounted in the proximal end and the first needle cannula is mounted in the distal end of the housing.

11. The medicated module of claim 10, where the two needle cannulae are not in fluid communication with the second medicament when the needle guard is fully extended in a distal direction, and where the two needle cannulae are in fluid communication with the second medicament when the needle guard is retracted in a proximal direction.

12. The medicated module according to claim 8, where the valve is a rotary valve which is configured to be rotated to enable fluid communication of the primary reservoir with the reservoir of the medicated module.

13. The medicated module according to claim 6, where the reservoir has a bypass which is adapted and arranged for bypassing the reservoir.

14. The medicated module of claim 13, wherein, when the needle guard is fully extended in a distal direction, the valve is configured to enable fluid communication of the needle cannulae with the bypass.

15. A drug delivery device to deliver two or more medicaments comprising, a housing comprising a primary reservoir of a first medicament containing at least one drug agent, the medicated module according to claim 6, wherein the medicated module is configured for fluid communication with the primary reservoir.

16. The drug delivery device of claim 15, wherein connection of the medicated module to the housing and retraction of the needle guard in a proximal direction triggers the time lock.

17. The drug delivery device of claim 15, wherein connection of the medicated module to the housing and retraction of the needle guard operates the valve.
